(19) United States
(12) Patent Application Publication
(10) Pub. No.: US 2020/0022898 A1
(43) Pub. Date: Jan. 23, 2020

(54) FILM FORMING COMPOSITIONS AND USES THEREOF

(71) Applicant: Coty Inc., New York, NY (US)

(72) Inventor: Yong ZHU, Marriottsville, MD (US)

(73) Assignee: Coty Inc., New York, NY (US)

(21) Appl. No.: 16/487,985

(22) PCT Filed: Jun. 29, 2018
(86) PCT No.: PCT/US2018/040341

§ 371 Date: Aug. 22, 2019

Related U.S. Application Data
(60) Provisional application No. 62/527,291, filed on Jun. 30, 2017.
(60) Provisional application No. 62/527,304, filed on Jun. 30, 2017.

(51) Int. Cl. A61K 8/81; A45D 34/04; A45D 44/00; C09D 133/06; C09D 139/06; C09D 133/26; C09D 175/04; A61Q 1/10; A61Q 19/00; A61Q 1/00; A61Q 5/00

(52) U.S. Cl. CPC A61K 8/8152; A45D 34/04; A45D 44/002; C09D 133/064; C09D 139/06; C09D 133/26; A61Q 5/00; A61K 8/8158; A61K 8/8182; A61Q 1/10; A61Q 19/00; A61Q 1/00; C09D 175/04

(57) ABSTRACT

The present disclosure is directed to film forming compositions comprising a first anionic or non-ionic non-crosslinking polyacrylate random copolymer and a second cationic non-crosslinking polyamide random copolymer. The present disclosure is also directed to methods of using the film forming compositions.

### FIELD OF THE INVENTION

The disclosure is directed, generally, to film forming compositions. These film forming compositions can be useful in a variety of applications, including use as a skin care product and a mascara product.

### BACKGROUND

A major unmet consumer need for most tightening products (e.g., skin tightening products) in the market is the poor capability to deliver long wearing benefits due to, among other things, (i) unmatched mechanical properties; (ii) insufficient adhesion to skin; and (iii) lack in robustness/compatibility with other cosmetic/skincare applications. Consequently, the tightening function of existing tightening products is minimized with repeated facial expressions or skin conditions (e.g., sweat, sebum, etc.). Film elongation becomes plastic and finally broken, in most cases leading to peeling/flaking.

### SUMMARY

The compositions of the various embodiments described herein address the shortcomings of existing tightening products.

A first embodiment of the disclosure, therefore, relates to a film forming composition comprising:
a first anionic, pseudo-cationic or non-ionic non-crosslinking polyacrylate random copolymer comprising the following monomer units:
at least one acrylate monomer, including acrylate, (meth)acrylate monomers or short-chain alkyl acrylate;
monomers having at least one carboxylic functional group selected from the group consisting of carboxylic esters, carboxylic acids, their salts, or precursors of carboxylate functions, and mixtures thereof and
a second cationic or pseudo-cationic non-crosslinking polyamide random copolymer comprising the following monomer units:
at least one amide monomer, including vinyl caprolactam monomers, vinylpyrrolidone monomers, and (meth)acrylamide or short-chain alkyl acrylamide monomers;
at least one cationic functional group, including quaternary amine or ammonium containing monomer and monomers having an amine functional group including primary, secondary, and tertiary amines;
wherein:
the film forming composition is a combination of first and second polymers; and if the second copolymer has a charge density of about 0.1 mEq/g to about 1 mEq/g, the first copolymer has a charge density of about −1 mEq/g to about −6 mEq/g or if the second copolymer has a charge density of about 1 mEq/g to about 6 mEq/g, the first copolymer has a charge density of about −0.1 mEq/g to about −1 mEq/g.

And a second embodiment relates to a two-component system comprising:
as a first component, a first anionic or non-ionic non-crosslinking polyacrylate random copolymer comprising the following monomer units:
at least one acrylate monomer, including acrylate, (meth)acrylate monomers or short-chain alkyl acrylate;
monomers having at least one carboxylic functional group selected from the group consisting of carboxylic esters, carboxylic acids, their salts, or precursors of carboxylate functions, and mixtures thereof.
as a second component separate from the first component, a second cationic non-crosslinking polyamide random copolymer comprising the following monomer units:
at least one amide monomer, including vinyl caprolactam monomers, vinylpyrrolidone monomers, and (meth)acrylamide or short-chain alkyl acrylamide monomers;
at least one cationic functional group, including quaternary amine or ammonium containing monomer and monomers having an amine functional group including primary, secondary, and tertiary amines.

### DESCRIPTION OF THE DRAWINGS

The drawings illustrate generally, by way of example, but not by way of limitation, various embodiments discussed herein.

FIG. 1 is an image of a Leneta card with lines indicating how to determine length for a contraction percentage measurement.

FIG. 2 is an image of an evenly contracted Leneta card with lines indicating how to determine length for a contraction percentage measurement.

FIG. 3 is an image of a non-evenly contracted Leneta card with lines indicating how to determine length for a contraction percentage measurement.

FIG. 4 is an image of a contracted Leneta card that has coiled with lines indicating how to determine length for a contraction percentage measurement.

FIG. 5 is images of skin treated with the compositions of the various embodiments described herein.

FIG. 6. is images of skin treated with the compositions of the various embodiments described herein, where the skin has been pretreated with an aqueous CaCl₂ solution and skin that was not pretreated with a an aqueous CaCl₂ solution.

### DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Disclosed herein are polymer combination systems that provide a more durable, high strength contraction force and fast dry profile when applied to a substrate. When used as skin smoothening/tensioning agent, the polymer combination system has improved tensioning of the substrate (e.g., skin) and durability against repeated movement (e.g., facial movement). When used as mascara and applied to the lashes, this produces desired eyelash curl and lift to gain lengthening appearance, and meanwhile undergoes fast solidification to keep the lash styling longer and durable.

Embodiments of the disclosure relate to film forming compositions comprising a first anionic, pseudo-cationic, or non-ionic non-crosslinking polyacrylate random copolymer comprising at least one acrylate monomer including acrylate, (meth)acrylate or short-chain alkyl acrylate monomers; monomers having at least one carboxylic functional group selected from the group consisting of carboxylic esters, carboxylic acids and their salts, or precursors of carboxylate functions, and mixtures thereof. The film forming composition also comprises a second cationic or pseudo-cationic non-crosslinking polyamide random copolymer comprising at least one amide monomer including vinyl caprolactam monomers, vinylpyrrolidone monomers, and (meth)acrylamide or short-chain alkyl acrylamide monomers; at least one cationic functional group including quaternary amine or ammonium containing monomer and primary, secondary, and tertiary amine functional groups.

Each of the at least one amide monomers is present in any suitable amount, such as from about 1% to about 10% by weight of the film forming compositions of the instant disclosure (e.g., from about 2% to about 8%, about 3% to about 6%, about 3% to about 7%, or about 2% to about 4% by weight of the film forming compositions of the instant disclosure).

Amide monomers include amide monomers with open-chain organic amide functional groups and derivatives having the formula:

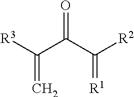

wherein R¹ is H, or C₁-C₅ alkyl or alkylene; R² is H, or C₁-C₁₈ alkyl or alkylene; and R³ is H or C₁C₅ alkyl, wherein the alkyl in R¹-R³ and alkylene groups in R² can be, independently, substituted, unsubstituted, linear or branched. Examples of amides include acrylamides and methacrylamides.

Commercially available examples of amide monomers include the monomers in copolymers including Styleze W, Styleze CC-10, AquaStyle 300 (PQ69), Aquaflex SF-40, ViviPrint 141, Conditioneze NT-20 all commercially available from Ashland Specialty Ingredients (ASI); and Ultrahold Strong, Luviset Clear, Luviquat Supreme (PQ68) all available from BASF. Other examples of copolymers comprising the amide monomers described herein can be found in Personal Care Product Consult Database (PCPC).

Amide monomers also include amide monomers with cyclic amide functional groups and derivatives having the formula:

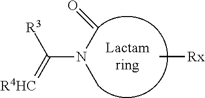

wherein the lactam ring can have 5 to 7 member rings (e.g. vinylpyrrolidones and vinyl caprolactams).

The lactam ring comprising the Rx group or groups are linked to one or more carbon atoms on the ring. The Rx group or groups are independently H or C₁-C₃₀ alkyl, which can be linear or branched; R³ is defined herein; and R⁴ is alkyl or alkylene, wherein the alkyl and alkylene groups can be, independently, substituted, unsubstituted, linear or branched. Amide monomers may contain one or more types of amide functional groups (e.g., open and/or cyclic amides, vinylpyrrolidones and/or vinyl caprolactams).

Commercially available examples of amide monomers include the monomers in copolymers including Copolymer 845, 937 and 958, Advantage LCA, LCE and S, PVP/VA (W635, 735), Gafquat, Aquaflex SF-40, Styleze W, Aquastyle 300, ViviPrint 141, Conditioneze NT-20, Styleze CC-10 all available from ASI; and Luviquat Supreme, Luviquat UltraCare, Luviquat Hold, Luviquat PQ11, Luviquat HM552, Luviquat Style, Luviquat FC, Luviquat Excellence, Luviset Clear all available from BASF. Other examples of this type of polymers can be found in Personal Care Product Consult Database (PCPC).

Amine monomers include those having the general formula:

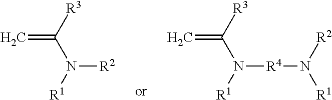

wherein R¹-R⁴ are defined herein.

Commercially available examples of amine monomers include the monomers in copolymers including Copolymer 845, 937 and 958, Advantage LCA, LCE and S, Gafquat, Aquaflex SF-40, Styleze W, Aquastyle 300, ViviPrint 141, Aquaflex XL-30, Styleze CC-10 all available from ASI; and Luviquat Supreme from BASF. Other examples of this type of polymers can be found in Personal Care Product Consult Database (PCPC).

Monomers also include amine monomers with quaternary ammonium functional groups and derivatives having the formula:

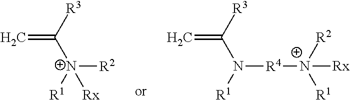

wherein R¹-R⁴, and Rx are defined herein and X is O or NR, wherein NR is a represents a secondary or tertiary amine.

Commercially available examples of amine monomers include the monomers in copolymers including Polyquaternium-5, -11, -14, -19, -22, -28, -37, -46, -47, -51, -55, -69, -87 (all available from BASF).

Still other monomers include monomers having the formula:

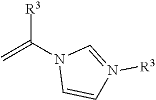

wherein R³ is defined herein and be present or absent. In addition, each R³ can be the same or different. When R³ is present, it should be understood that the nitrogen to which R³ is attached bears a positive charge and there is a counterion involved.

Commercially available examples of quaternary amine monomers include the monomers in copolymers including Polyquaternium-16, -44, -46, -68 (available from BASF).

Monomers also include amide or acrylate monomers with quaternary ammonium functional groups and derivatives having the formula:

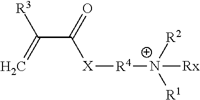

wherein R¹-R⁴, and Rx are defined herein and X is O or NR, wherein NR is a represents a secondary or tertiary amine.

Commercially available examples of such monomers include the monomers in copolymers including Polyquaternium-4, -5, -7, -8, -9, -11, -12, -13, -18, -28, -33, -36, -37, -45, -47, -49, -52, -53, -55, -63, -64, -68, -69, -85, -89, -91, -109, and others as described PCPC.

Still other monomers include monomers with carboxylic acid, salt and ester functional groups and derivatives having the formula:

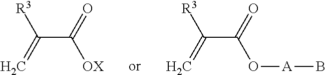

wherein X is H, or ionizable metal ions; A is C₁-C₈ alkyl or alkylene group, wherein the alkyl or alkylene is linear or branched; B is H, amide, imide, amine, quaternized ammonium, or acid-ester/acid/salt, or their derivatives, wherein the amine functional groups are primary, secondary, and tertiary amines; open and cyclic amines; and R³ is defined herein.

In some embodiments, monomers include: acrylates, methacrylates, acrylic acids and their salts, methacrylic acids and their salts. The carboxylate monomers may include precursors of carboxylate functions, such as tert-butyl (meth)acrylates, alkyl-2-amino ethyl esters of (meth)acrylates which give rise to carboxylic functions by hydrolysis (under more stressed pH, temperature conditions, in the presence of catalysts, or other approaches). Commercially available examples of copolymers containing such monomers include Advantage Plus, LCA, LCE and S, W635 and 735, Copolymer 845, 937 and 958, Aquaflex XL-30, PVP/VA E-735, E-635, E-535 and W-735, Gafquat, Allianz OPT from ASI; Luviquat PQ11, UltraHold Strong, Luviset Shape, Luviset One, Luviflex Soft, Cosmedia SP from BASF. Other examples of this type of polymers can be found in Personal Care Product Consult Database (PCPC).

The ester/acid/salt/anhydride functional groups may contain one or more types of esters/acids/salts/anhydrides (e.g. esters, acids and/or salts).

While not wishing to be bound by any specific theory, it is believed that some criteria for achieving the surprising, significant, and unexpected high contraction and high durability (e.g., recovery of a film after repeated and/or exaggerated facial stress) of the film forming compositions of the various embodiments described herein is the charge density, charge dissimilarity, and structure dissimilarity of the polymers. It is believed, for example, that the improved durability of the film forming compositions may be driven, at least in part, by a gel network structure formed during electrostatic-induced phase transition. The gel system is believed to provide high flexibility and strong film adhesion to substrate surfaces, including the skin. Thus, for example, the first copolymer and the second copolymers of the film forming compositions comprise less than 40% monomers have the same chemical structures, for example have from about 0% to about 39% of the same monomers. In another embodiment, the first copolymer and the second copolymers have from about 0% to about 20% of the same monomers. In another embodiment, the first copolymer and the second copolymers have from about 0% to about 10% of the same monomers. In yet other embodiments, the first copolymer and the second copolymer have from about 0% to about 5% of the same monomers.

In some embodiments, the second copolymer has one or more quaternary ammonium or other cationic side chains. In some embodiments, the second polymer comprises from about 0.1 to about 45 percent; or about 1 to about 20 percent of the quaternary amine or ammonium or other cationic side chains.

In other embodiments, the first copolymer has one or more carboxylate or other anionic side chains. In some embodiments, the first polymer comprises from about 0.1 to about 45 percent or about 1 to about 30 percent of the carboxylate or other anionic side chains.

In one embodiment of the film forming composition, the ratio of the first copolymer to the second copolymer is from about 1:50 to about 50:1. In another embodiment, the ratio of the first copolymer to the second copolymer is from about 1:10 to about 10:1. In yet another embodiment, the ratio of the first copolymer to the second copolymer is about 1:5 to about 5:1. In another embodiment, the ratio of the first copolymer to the second copolymer is about 1:2 to 2:1.

In one embodiment of the film forming composition, the amount of total polymers in the composition is from about 1% to about 30% (e.g., about 5 to about 10%; about 10% to about 20%; about 15% to about 25%; or about 13% to about 20%).

In one embodiment of the film forming composition, the first and the second copolymer have a combined polymer solid level greater than about 1% by weight of the composition (w/w). In another embodiment of the film forming composition, the first copolymer to the second copolymer have a combined total polymer solid level greater than about 2% by weight of the composition (w/w). In another embodiment, the first copolymer to the second copolymer have a combined total polymer solid level greater than about 5% by weight of the composition (w/w). In another embodiment, the first copolymer to the second copolymer have a combined polymer solid level from about 2% to about 50% by weight of the composition (w/w). In yet another embodiment, the first copolymer to the second copolymer have a combined polymer solid level from about 5% to about 30% by weight of the composition (w/w).

In one embodiment of the film forming composition, the first copolymer to the second copolymer are random copolymers and include water soluble copolymers having molecular weight MW of greater than about 10,000 as measured by light scattering methods. In another embodiment, the water soluble copolymers have molecular weight MW from 20,000 to 4,000,000. In another embodiment, the water soluble copolymers have molecular weight MW from 50,000 to 2,000,000.

The second copolymer in the film forming compositions described herein is cationic (e.g., containing quaternary ammonia or other permanent cationic ions), pseudo-cationic or pH dependent cationic (e.g., containing primary, secondary and tertiary amines). The first copolymer in the film forming compositions described herein is anionic (e.g., containing carboxylic ions or other anionic ions), pseudo-anionic or pH dependent anionic (e.g; containing carboxylic acid, carboxylic ester or hydrolysable/degradable ester), or precursor of anionic functions, amphoteric, or zwitterionic in charges.

The charge density (cationic or anionic) of the polymers is expressed in milliequivalent per gram (mEq/g), defined theoretically as the number of the equivalent charged groups per gram of polymer mass. The theoretical charge density may be determined based on the contents of charged monomers in the polymers.

The apparent or relative charge density or surface charge density of water soluble or water dispersible polymers and/or particles with hydrodynamic diameter or mean particle size of 10-1000 nm in size may be determined or supported by zeta-potential, streaming potential, or streaming current.

In one embodiment, the second copolymer has a positive charge density from about 0.1 to about 6.0 milliequivalents/gram (mEq/g) at pH 5-9. In another embodiment, the first copolymer has a positive charge density from about 0.3 to about 4.0 mEq/g at pH 5-9. In another embodiment, the first copolymer has a positive charge density from about 0.4 to about 0.8 mEq/g at pH 5-9.

In another embodiment, the first copolymer has a negative charge density about 0.0 to about −6.0 mEq/g based on anionic monomer contents. In another embodiment, the first copolymer has a negative charge density from less than 0.0 to about −4.0 mEq/g based on anionic monomer contents. In another embodiment, the first copolymer has a negative charge density from less than 0.0 to about −1.0 mEq/g based on anionic monomer contents.

In some embodiments, if the second copolymer has a charge density of about 0.1 mEq/g to about 1 mEq/g, the first copolymer has a charge density of about −1 mEq/g to about −6 mEq/g. Or if the second copolymer has a charge density of about 1 mEq/g to about 6 mEq/g, the first copolymer has a charge density of about −0.1 mEq/g to about −1 mEq/g.

In one embodiment, the film forming compositions of the various embodiments described herein produces a contraction when applied to a Leneta card. When the composition is applied to a Leneta card, the card has a minimum contraction of about 5% to about 10% with minimum synergy of about 120% to about 160% (as described by the “Contraction Test” method herein) when the Leneta card is kept at a temperature in the range of 22 to 28° and at a relative humidity in the range of about 40% to 50% to measure the contraction.

In one embodiment, the film forming compositions of the various embodiments described herein can be used as a dry film or as a powder. These dry forms of the film forming compositions can be applied to a wetted surface or wetted after they are applied to a surface to deliver mechanical benefits to lashes and/or skin. The shrinkage benefits that are obtained from the film forming compositions are capable of being rejuvenated with water exposure, enabling benefits to cycle from wet to dry to wet to dry while still maintaining the technical polymer benefit.

For lashes, dry film and/or powder forms of the film forming compositions may be placed on wetted lashes or lashes that are wetted after film deposition to produce lash lift and curl. For skin, powder and/or film forms can be applied to pre or post wetted areas of skin to enable skin tensing effects to impact fine lines and wrinkles. In another embodiment, the film forming compositions may be used as a combination of dry film, powder and/or wet formulation (e.g., in water or ethanol).

In one embodiment, the dry film and powders of the present disclosure can be used for targeted applications on lashes or skin. For lashes, dry formulations could be applied and then wetted to produce different lash looks from lash lift, curl and lash lift and curl depending on where formulation has been applied. For example, the powder could be selectively applied to the lash only near the eyelid, the center of the lash, or the tip of the lash. For skin, dry formulations could be applied to wet skin, or applied and then wetted, on target areas, like the “crow's feet” area or the forehead, to enable targeted skin tensing effects to impact fine lines and wrinkles.

In one embodiment, for targeted application on lashes, a modified eyelash curling device could be used. In such a modified device, the rubber-like strip is replaced with a powder or film delivery mechanism. In another embodiment, the delivery mechanism is a trough filled with powder.

The film forming composition of the of the various embodiments described herein can be used in a variety of applications, including as a mascara formulation, a hair styling product, a skin care product, a skin foundation product, a lip product, a nail care product, and a kit. Embodiments also contemplate a water based system comprising the film forming composition.

The compositions disclosed herein may be used in many end-use applications. Examples include (but are not limited to) a water phase suspension, an oil in water emulsion, a water in oil emulsion, a silicone in water emulsion, a water in silicone emulsion, a Pickering emulsion, and/or an oil phase suspension, and/or kits.

The film forming compositions of the various embodiments described herein can comprise additives, in addition to the first copolymer and the second copolymer, regardless of the application (e.g., mascara formulation, a hair styling product, a skin care product, a skin foundation product, a lip product, a nail care product, or a kit). The additives include, but are not limited to, buffering agents, carriers; oils typically used in cosmetics; colorants; thickeners; biological, chemical, nutraceutical, or pharmaceutical active, or a combination thereof; additional polymers; fats; plasticizers; plasticizing solvents; rheology modifiers; oil-soluble or dispersible additives; keratin adhesive and deposition aids; reinforcement fillers; and combinations of two or more additives.

Suitable carriers include volatile carriers that quickly volatilize from the surface of a substrate, leaving the desired components behind. The volatile carrier may be present at about 1% to about 85%, about 2% to about 85%, about 2% to about 15%, about 2% to about 5%, about 1% to about 3%, about 5% to about 10%, about 10% to about 80%, or even about 20% to about 70% by weight based on the weight of the composition. Non-limiting examples of suitable volatile carriers include volatile hydrocarbons, volatile alcohols (e.g., ethanol), volatile silicones, and mixtures thereof.

Suitable buffering agents include amine-based buffering agents, such as 2-amino-2-methyl-1-propanol, which may be referred to herein as “aminomethyl propanol.” Buffering agents, when present, can each be present in any suitable amount, including from about 0.01% to about 2% by weight of the film forming compositions of the instant disclosure (e.g., about 0.05% to about 0.5%, about 0.01% to about 0.05%, about 0.05% to about 0.1%, 0.09% to about 0.2%, about 0.09% to about 0.15% or about 0.05% to about 0.3% by weight of the film forming compositions of the instant disclosure).

The film forming compositions of the instant disclosure can have any suitable pH, such as a pH of from about 3 to about 9 (e.g., about 4 to about 7, about 5 to about 8, about 6 to about 8, about 7 to about 9 or about 7 to about 8). And a buffering agent can be used to maintain a pH from about 3 to about 9 (e.g., about 4 to about 7, about 5 to about 8, about 6 to about 8, about 7 to about 9 or about 7 to about 8).

The film forming compositions of the instant disclosure can have any suitable viscosity at 25° C. measured using a Brookfield-type viscometer, such as a viscosity of from about 1000 mPa to about 10,000 mPa (e.g., about 1000 to about 5000, about 1500 to about 4000, about 2000 to about 5000, about 2000 to about 4000 or about 1000 to about 3000).

Hydrocarbon oils suitable for use as a carrier include those having boiling points in the range of 60-260° C., such as hydrocarbon oils having a carbon chain length of from C8 to C20 (e.g., C8 to C20 isoparaffins). Particularly suitable examples of isoparaffins include those selected from the group consisting of isododecane, isohexadecane, isoeicosane, 2,2,4-trimethylpentane, 2,3-dimethylhexane and mixtures thereof. Isododecane is available from Presperse under the brand name Permethyl 99A. Alcohols suitable for use may include C₁-C₄ monoalcohols, such as ethyl alcohol and isobutyl alcohol.

A volatile silicone fluid may also be used as a carrier herein. Suitable volatile silicone fluids include dimethicone, trimethicone, and cyclomethicones. Nonlimiting examples of commercially available volatile silicones include 244 Fluid, 344 Fluid and 245 Fluid, and/or 345 Fluid from Dow Corning Corporation.

Oils typically used in cosmetics include those selected from the group consisting of polar oils, non-polar oils, volatile oils, non-volatile oils and mixtures thereof. These oils may be saturated or unsaturated, straight or branched, aliphatic or aromatic hydrocarbons. Preferred oils include non-polar volatile hydrocarbons including isodecane (such as Permethyl-99A®, available from Presperse Inc.) and the C₇-C₈ through C₁₂-C₁₅ isoparaffins (such as the Isopar® Series available from Exxon Chemicals).

Non-polar, volatile oil may be included in the cosmetic composition to impart desirable aesthetic properties (e.g., good spreadability, non-greasy and/or tacky feel, quick drying to allow pigment particles to set on skin) to a composition. Non-polar, volatile oils suitable for use herein include silicone oils; hydrocarbons; and mixtures thereof. The non-polar, volatile oils may be either saturated or unsaturated, have an aliphatic character and be straight or branched chains or even contain alicyclic or aromatic rings. Examples of suitable non-polar, volatile hydrocarbons for use herein include polydecanes such as isododecane and isodecane (e.g., Permethyl-99A which is available from Presperse Inc.), dodecanes and tetra dodecanes (such as Parafol 12-97 and Parafol 14 from Sasol), and the C₇-C₈ through C₁₂-C₁₅ isoparaffins (such as the Isopar Series available from Exxon Chemicals). Examples of non-polar, volatile liquid silicone oils are disclosed in U.S. Pat. No. 4,781,917, which is incorporated by reference as if fully set forth herein. Additionally, a description of various volatile silicone oils may be found in Todd et al., “Volatile Silicone Fluids for Cosmetics”, Cosmetics and Toiletries, 91:27-32 (1976). Suitable volatile silicone oils include cyclic volatile silicones of the formula:

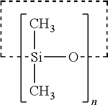

wherein n is from about 3 to about 7; and linear volatile silicones corresponding to the formula:

(CH₃)₃SiO[Si(CH₃)₂O]ₘSi(CH₃)₃

wherein m is from about 0 to about 7.

Linear volatile silicone oils generally have a viscosity of less than about 5 centistokes at 25° C., whereas the cyclic silicones have viscosities of less than about 10 centistokes at 25° C. Examples of suitable volatile silicone oils include cyclomethicones of varying viscosities, e.g., Dow Corning 200, Dow Corning 245, available from Dow Corning Corp.); SF-1204 and SF-1202 Silicone Fluids (commercially available from Momentive Specialty Chemicals), and SWS-03314 (commercially available from Wacker Chemie AG.). In addition, Caprylyl Methicone such as Dow Corning FZ3196 can be used. Other examples of non-polar, volatile oils are disclosed, for example, in Cosmetics, Science, and Technology, Vol. 1, 27-104 edited by Balsam and Sagarin, 1972.

Colorants include, but are not limited to, dyes, pigments, lakes, and mixture thereof. (e.g., organic or inorganic pigments and colorants approved for use in eye-area cosmetics by PCPC and/or the FDA.) Exemplary inorganic pigments include particles of iron oxides (e.g., yellow, brown, red, black), titanium dioxides, iron sulfides, ultramarines, chromium oxides (e.g., green) or other conventional pigments used in cosmetic formulations. Examples of organic pigments include D&C Black No. 2, D&C Black No. 3, FD&C Red No. 40, D&C Green No. 5, FD&C Blue No. 1, and FD&C Yellow No. 5. Examples of lake dyes include various acid dyes which are laked with aluminum, calcium or barium. Additional colorants for use herein include annatto, caramel, carmine, β-carotene, bismuth oxychloride, ferric ammonium ferrocyanide, ferric ferrocyanide, chromium hydroxides (e.g., green), guanine, mica, aluminum powder, bronze powder, copper powder, manganese violet, zinc oxide. Suitable colorants along with their chemical structure are described in, e.g., 21 C.F.R. Part 74 and in the PCPC Cosmetic Ingredient Handbook, (1988), published by the Cosmetics, Toiletry and Fragrances Association, Inc. Other colorants may also be used as they are developed and determined safe.

Encapsulated colorant microparticles having average diameters of 0.1 to 50 microns may be acceptable for use in mascara compositions. Suitable examples of encapsulated colorant microparticles are disclosed in copending U.S. Publication Nos. 20090263658 and 20090271932A1, both of which as incorporated by reference as if fully set forth herein. The encapsulated colorant microparticles may comprise from 1 to 60% by weight of at least one colorant, for example 5% to 40% or even 6% to 25% by weight. Microencapsulated colorants may provide a more vibrant color to products used around the eye area, including eyelashes. The primary colors are understood to mean red, yellow and blue.

In one embodiment, the film forming compositions comprise from about 0.1 to about 70% by weight, for example from about 0.5 to about 50% by weight, and from about 1.0 to about 35% by weight based on the total weight of the composition, of a colorant. Colorants in the form of particles and/or encapsulants having average diameters of 0.1 to 50 microns may be acceptable for use in the present compositions. In another embodiment, the particles have average diameters of 0.1 to 10 microns. In another embodiment, the particles have average diameters of 0.1 to 5 microns. It may be desirable to select colorant particles with a diameter that is less than the thickness of the mascara composition dried-down film. The small size of the colorant particles may allow them to be fully encased in the dried film.

The film forming compositions of the various embodiments described herein can include thickeners. The compositions can be thickened or structured with colloidal particles and/or waxes.

Thickening agents that may be useful include carboxylic acid polymers such as the carbomers (e.g., the CARBOPOL® 900 series such as CARBOPOL® 954 by Lubrizol). Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, PEMULEN TR-1, and PEMULEN TR-2, from Lubrizol.

Additional suitable thickening agents include the polyacrylamide polymers and copolymers. An example of such polymers is the polyacrylamide polymer has the CTFA designation “polyacrylamide and isoparaffin and laureth-7” and is available under the trade name SEPIGEL 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson, N.J.). Other suitable thickening agents useful herein are sulfonated polymers such as the CTFA designated sodium polyacryloyldimethyl taurate available under the trade name Simulgel 800 from Seppic Corp. and Viscolam At 100 P available from Lamberti S.P.A. (Gallarate, Italy). Another commercially available material comprising a sulfonated polymer is Sepiplus 400 available from Seppic Corp.

Waxes may be useful as thickeners and/or as structuring agents including natural, synthetic, and surface modified waxes, including cold water process wax (such as CPW brands by JEEN International Corp). Waxes are defined as lower-melting organic mixtures or compounds of high molecular weight, solid at room temperature and generally similar in composition to fats and oils except that they contain no glycerides. Some are hydrocarbons, others are esters of fatty acids and alcohols. Waxes useful in the compositions of the present disclosure include animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof wherein the waxes have a melting point between 55° and 100° C. and a needle penetration value, as measured according to the American standard ASTM D5, of 3 to 40 units at 25° C. The principle of the measurement of the needle penetration according to the standards ASTM D5 consists in measuring the depth, expressed in tenths of a millimeter, to which a standard needle (weighing 2.5 g and placed in a needle holder weighing 47.5 g, i.e. a total of 50 g) penetrates when placed on the wax for 5 seconds. Waxes are used at levels in order to provide sufficient bulk material that resists drying out after application, providing thickness to the lashes.

Waxes may be useful to maintain the film durability of the mascara composition. In some instances, the mascara composition may include from 0.1-15% wax In another embodiment, the mascara composition may include from 1-10% wax. In another embodiment, the mascara composition may include from 4-8% wax. In some instances, it may be desirable to include wax at an amount of less than 3.0%, for example, less than about 1.0% or even less than 0.1%, by weight, of wax and wax-like components. In some instances, the present mascara composition is free of wax.

Specific waxes that may be useful include beeswax, lanolin wax, shellac wax (animal waxes); carnauba, candelilla, bayberry (vegetable waxes); ozokerite, ceresin, (mineral waxes); paraffin, microcrystalline waxes (petroleum waxes); polyethylene, (ethylenic polymers); polyethylene homopolymers (Fischer-Tropsch waxes); C24-45 alkyl methicones (silicone waxes); and mixtures thereof. Most preferred are beeswax, lanolin wax, carnauba, candelilla, ozokerite, ceresin, paraffins, microcrystalline waxes, polyethylene, C24-45 alkyl methicones, and mixtures thereof.

Clays may be useful to provide structure or thickening. Suitable clays can be selected, e.g., from montmorillonites, bentonites, hectorites, attapulgites, sepiolites, laponites, silicates and mixtures thereof. Suitable water dispersible clays include bentonite and hectorite (such as Bentone EW, LT from Rheox); magnesium aluminum silicate (such as Veegum from Vanderbilt Co.); attapulgite (such as Attasorb or Pharamasorb from Engelhard, Inc.); laponite and montmorillonite (such as Gelwhite from ECC America); and mixtures thereof.

Disteardimonium hectorite is a suitable thickener to build structure/viscosity in the present mascara composition. This enables proper spreading/deposition across lashes, and ensures adequate stability/suspension of colorant particles in dispersion over time. It is preferable that the diameter of the disteardimonium hectorite is smaller than the thickness of the mascara composition dried-down film. The preferred diameter of the disteardimonium hectorite is less than 10 microns. The mascara compositions may comprise from about 1% to about 25% of suitable thickener such as disteardimonium hectorite, from about 2% to about 20%, or even from about 3% to about 15%. Suitable thickening agents also include cellulose and modified cellulosic compositions such as, carboxymethyl cellulose, hydroxyethylcellulose, cellulose acetate propionate carboxylate, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers some portion of the hydroxy groups of the cellulose polymer are hydroyxalkylated (preferably hydroxyethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Examples of alkyl groups useful herein include those selected from the group consisting of stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the PCPC designation cetyl hydroxyethylcellulose, which is the ether of cetyl alcohol and hydroxyethylcellulose. This material is sold under the tradename Natrosol® CS Plus from ASI.

The film forming compositions of the various embodiments described herein can comprise a safe and effective amount of a biological, chemical, nutraceutical, or pharmaceutical active, or a combination thereof. Biological actives may include prostaglandins, antimicrobials, antibacterials, biocides, preservatives, proteins, amino acids, peptides, hormones, growth factors, enzymes (e.g., glutathione sulphydryl oxidase, transglutaminase), therapeutics, oligonucleotides, genetic materials (e.g., DNA, RNA), and combinations thereof. Chemical actives may include dyes, surfactants, sensates, hair conditioners, hair dyes, hair growth agents, hair styling gels, and combinations thereof. Nutraceutical actives may include proteins, preservatives, vitamins, food-additive materials, and combinations thereof. Pharmaceutical actives may include antibiotics, drugs, hair growth agents, and combinations thereof. Specific biological actives include, but are not limited to BioBenefity, MandarinClear, Ougon Liquid B, Neoppro EW, Biopolymer SA-N, butylene glycol, and combinations of two or more biological actives. Biological actives, when present, can each be present in any suitable amount, including from about 0.1% to about 5% by weight of the film forming compositions of the instant disclosure (e.g., about 1% to about 5%, about 1% to about 3%, about 0.5% to about 2%, 1% to about 2%, about 0.3% to about 1% or about 0.5% to about 1% by weight of the film forming compositions of the instant disclosure).

In addition to the first copolymer and the second copolymer, the composition may also include additional polymers, including additional water-soluble film forming polymers. In one embodiment, water-soluble, film forming polymers comprise from about 0.1% to about 50%, from about 1% to about 40%, from about 3% to about 30% of the composition or about 0.1% to about 3% of the composition.

The additional polymers comprise polymers formed from monomers, said monomer derivatives, mixtures of said monomers, mixtures of said monomer derivatives, natural polymers and mixtures thereof. The film forming polymers disclosed herein also include chemically modified versions of the polymers disclosed herein. Said monomers are selected from the group consisting of olefin oxides, vinyl pyrrolidone, vinyl caprolactam, vinyl esters, vinyl alcohols, vinyl cyanides, oxazilines, carboxylic acids and esters and mixtures thereof. Examples of vinyl pyrrolidone polymers include polyvinylpyrrolidone, vinyl acetate/vinyl pyrrolidone copolymer and mixtures thereof. Examples of polyvinyl esters include vinyl acetate/crotonic acid copolymer, vinyl acetate crotonic acid vinyl neodecanoate copolymer and mixtures thereof. Examples of vinyl alcohol include vinyl alcohol vinyl acetate, vinyl alcohol/poly(alkyleneoxy)acrylate, vinyl alcohol/vinyl acetate/poly-(alkyleneoxy)acrylate and mixtures thereof. Examples of olefin oxides include polyethylene oxide, polypropylene oxide and mixtures thereof. Preferred polycarboxylic acids and their esters are selected from the group consisting of acrylates, acrylates/octylacrylamide copolymers and mixtures thereof. The preferred oxaziline is polyoxaziline.

The additional polymers which may be useful comprise natural polymers selected from the group consisting of cellulose derivatives, algin and its derivatives, starch and its derivatives, guar and its derivatives, shellac polymers and mixtures thereof. Examples of cellulose derivatives include hydroxyethylcellulose, hydroxypropylcellulose, hydroxypropyl methylcellulose, ethylhydroxyethyl cellulose and mixtures thereof.

Other additional polymers include elastic polymers such as BAYCUSAN® C1000, C1003, C1004, C1008, C2000, Luviset PUR, Si-Tec RE100, Gransil SiW -066, Polynecon PPI-SA-15D, Polyderm PPI-SA, Polynecon PPI-SI-WS, Polyderm PE-PA, FA4103 silicone acrylate emulsion, and combinations of one or more of these polymers. Additional polymers, when present, can each be present in any suitable amount, including from about 0.5% to about 10% by weight of the film forming compositions of the instant disclosure (e.g., about 1% to about 5%, about 0.5% to about 1%, about 1% to about 3%, 3% to about 8% or about 5% to about 10% by weight of the film forming compositions of the instant disclosure).

Fats, when included in the film forming compositions of the various embodiments described herein, include fats derived from animals, vegetables, synthetically derived fats, and mixtures thereof wherein said fats have a melting point from about 55° Cto about 100° C. and a needle penetration value, as measured according to the American standard ASTM D5, from about 3 to about 40 units at 25° C. Preferably the fats selected for use in the compositions of the present disclosure are fatty acid esters which are solids at room temperature and exhibit crystalline structure. Examples of fatty acid esters useful in the compositions of the present disclsoure include the glyceryl esters of higher fatty acids such as stearic and palmitic such as glyceryl monostearate, glyceryl distearate, glyceryl tristearate, palmitate esters of glycerol, C₁₈-C₃₆ triglycerides, glyceryl tribehenate and mixtures thereof.

Plasticizing solvents suitable for use herein are slow-evaporating, water-miscible or dispersible cosolvents that are 1) generally recognized as safe or 2) include slow evaporating glycols and glycol ethers, such as propylene glycol; butylene glycol; hexylene glycol; dipropylene glycol; dipropylene glycol methyl ether (commonly known as DPM); propylene glycol phenyl ether; and polyethylene glycols (PEGs) such as PEG 4 and PEG 8. Other exemplary plasticizing solvents include propylene carbonate, dimethyl isosorbide, and mixtures thereof. A wide variety of plasticizing solvents are listed in the CTFA International Cosmetic Ingredient Dictionary and Handbook, 3rd Ed., Cosmetic and Fragrance Assn., Inc., Washington D.C. (1982) pp. 575-580. The plasticizing solvent may be present in amounts of from 0.0% to 30% or even 5% to 20%, and generally appear in a ratio of solvent to polymer of from 10:1 to 1:5 or even 4:1 to 1:2. The plasticizing solvent is chosen to provide for water co-solvency, suitable solubility regarding the polymer, low volatility, stability, and safety (i.e., lack of toxicity). Thus, the cosmetic composition herein employs safe solvents that provide little or no sensation of tackiness or cooling (usually due to evaporation) on the applied area.

The plasticizing solvent may be chosen such that the polymer and plasticizing solvent are formulated in the aqueous phase of the emulsion, which may help reduce any tacky sensation of polymer contacting the users hands and fingers during application of the cosmetic composition. Because the solvent exhibits a slow evaporation rate and is present in the aqueous phase, it helps extend the workability of the mascara and delays any perceived onset of tackiness for up to two minutes.

Rheology modifiers that may be useful include both associated and non-associated thickeners, including alkaline swellable, hydrophobic modified, polyurethane type thickeners and structuring agents. Useful rheology modifiers include natural gums and extracts, modified (semi-synthetic) gums and extracts, hydrophilic natural and synthetic silicate and clay mineral agents, hydrophobic silicas, inorganic and polymeric porous microparticle absorbents, synthetic polymers (such as acrylic polymers), and mixtures thereof.

Natural gums and extracts for use in the compositions of the present disclosure include, but are not limited to, plant exudates, such as gum arabic, gum tragacanth, gum karaya, and gum ghatti; plant extracts, such as pectins; plant seed flours or extracts, such as locust bean gum, guar gum, psyllium seed gum, and quince seed gum; seaweed extracts, such as agar, alginates, and carrageenans; seed starches, such as corn starch, wheat starch, rice starch, and sorghum starch; tuber starches, such as tapioca starch and potato starch; animal extracts, such as gelatin and caseinates; and mixtures thereof.

Modified (semi-synthetic) gums and extracts for use in the compositions of the present disclosure include, but are not limited to, cellulose derivatives, such as sodium carboxymethylcellulose, hydroxyethylcellulose, hydroxypropylcellulose, methylcellulose, and hydroxypropyl methylcellulose, as well as alkyl-modified cellulose derivatives, such as cetyl hydroxyethylcellulose; modified plant extracts, such as hydroxypropyl guar; microbial or biosynthetic gums, such as xanthan gum, sclerotium gum, gellan gum, dextran and its derivatives; modified starches and starch derivatives, such as potato starch modified, corn starch modified, hydroxypropyl starch, dextrin and its derivatives; modified animal derivatives, such as chitin or chitosan, and their derivatives, collagen derivatives; and mixtures thereof.

Hydrophilic natural and synthetic clay mineral agents for use in the compositions of the present disclosure include, but are not limited to, hectorites, such as those sold under tradenames BENTONE® (Elementis Specialties); bentonites and montmorillonites, such as those sold under tradenames OPTIGEL®, GELWHITE® and MINERAL COLLOID® (by BYK Additives & Instruments), and POLARGEL® (AMCOL Health & Beauty Solutions); magnesium aluminum silicates, such as those sold under tradenames VEEGUM® (R. T. Vanderbilt Company), MAGNABRITE® (AMCOL Health & Beauty Solutions), and GELWHITE® MAS (BYK); sodium magnesium silicate, such as those sold under tradenames OPTIGEL® SH and LAPONITE® (both by BYK); lithium magnesium sodium silicate, such as LUCENTITE® SWN (Kobo Products); lithium magnesium silicate, such as LUCENTITE® SAN (Kobo Products); and mixtures thereof.

Hydrophobic silicas include hydrophobically modified fumed silicas, such as WACKER HDK® H15, H20, and H30 (Wacker-Chemie), and hydrophobic grades under tradenames of AEROSIL® (e.g., R816; Degussa AG) and CAB-O-SIL® (Cabot Corporation); and mixtures thereof. Hydrophobic silicas, when present, can each be present in any suitable amount, including from about 0.01% to about 2% by weight of the film forming compositions of the instant disclosure (e.g., about 0.1% to about 0.5%, about 0.1% to about 0.3%, about 0.05% to about 0.5%, 0.01% to about 0.05% or about 0.01% to about 0.03% by weight of the film forming compositions of the instant disclosure).

Inorganic and polymeric porous microparticle absorbents include high porosity/void volume fumed silicas, such as MSS-5003H and Silica Shells (both sold by Kobo Products), high porosity/void volume silicates like calcium silicate, such as sold under tradename HUBERDERM™ (J. M. Huber Corporation); high porosity/void volume polymeric particle absorbents including methacrylate polymers like allyl methacrylates copolymer, sold as POLY-PORES E-200 (AMCOL Health & Beauty Solutions), and cross-linked dimethacrylate copolymers like lauryl methacrylate/glycol dimethacrylate crosspolymer sold as POLYTRAP® 6603 (Enhanced Derm Technologies); high porosity cellulose beads like Cellulobeads® (Kobo Products); and mixtures thereof.

Synthetic polymers include, but are not limited to, acrylic polymers, such as polyacrylates and polymethacrylates, and acrylic copolymers and crosspolymers, such as the carbomers or acrylates/C10-C30 alkyl acrylate crosspolymers sold under tradename CARBOPOL® (Lubrizol), and sodium polyacrylate sold under tradename RAPITHIX™A-100 (ASI); acrylic acid/vinylpyrrolidone crosspolymers sold under tradename ULTRATHIX™P-100; alkali-soluble/swellable emulsion (ASE) polymers, hydrophobically-modified alkali-soluble/swellable emulsion (HASE) polymers, and hydrophobically-modified ethoxylated urethane (HEUR) polymers, such as those sold under tradename ACULYN™ (Dow Chemical Company) and STRUCTURE® (Akzo Nobel Company); hydrophobically-modified ethoxylate urethane alkali-soluble/swellable emulsion (HUERASE) polymers, such a those sold under tradename UCAR® POLYPHOBE® (Dow Chemical Company); copolymers of methyl vinyl ether and maleic anhydride, such as PVM/MA decadiene crosspolymer sold under tradename STABILEEZE® (ASI); hydrophobically modified non-ionic associative thickeners such as those sold under tradename PURE-THIX® (BYK); and mixtures thereof. Synthetic polymers, when present, can each be present in any suitable amount, including from about 0.05% to about 2% by weight of the film forming compositions of the instant disclosure (e.g., about 0.1% to about 2%, about 0.1% to about 0.5%, about 0.1% to about 1%, 1% to about 2% or about 0.5% to about 1% by weight of the film forming compositions of the instant disclosure).

The choice of oil-soluble or dispersible additive and the amount can depend on the intended use of the composition and the effectiveness of the compound. In semi-permanent mascara, top coat and remover compositions, the oil-soluble or dispersible additive chosen is acceptable for skin and eye contact, as is well known to the skilled formulator. Suitable oil-soluble or dispersible additives are incorporated at levels generally between 1 and 20% by weight based on the weight of the matrix bead (equivalent to 90 to 300% on weight of the colorant). Preferably 5 to 15% by weight of the oil-soluble or dispersible additive is employed.

The oil-soluble or dispersible additive may include fatty alcohols such as Guerbet alcohols based on fatty alcohols having from 6 to 30, preferably from 10 to 20 carbon atoms including lauryl alcohol, cetyl alcohol, stearyl alcohol, cetearyl alcohol, oleyl alcohol, benzoates of C₁₂-C₁₅ alcohols, acetylated lanolin alcohol, etc. Especially suitable is stearyl alcohol. The oil-soluble or dispersible additive may include fatty acids such as Linear fatty acids of C₆-C_{24,} branched C₆-C₁₃ carboxylic acids, hydroxycarboxylic acids, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids). Further components that can be used are dicarboxylic acids of C₂-C₁₂, such as adipic acid, succinic acid, and maleic acid. Aromatic carboxylic acids, saturated and/or unsaturated, especially benzoic acid, can be used. Additional components that can be used as the oil soluble or dispersible additive include carboxylic acid salts; alkaline soaps of sodium, potassium and ammonium; metallic soaps of calcium or magnesium; organic basis soaps such as lauric, palmitic, stearic and oleic acid, etc., alkyl phosphates or phosphoric acid esters: acid phosphate, diethanolamine phosphate, potassium cetyl phosphate.

Other useful oil-soluble or dispersible additives comprise mild surfactants, super-fatting agents, consistency regulators, additional thickeners, polymers, stabilizers, biologically active ingredients, deodorizing active ingredients, anti-dandruff agents, film formers, swelling agents, UV light-protective factors, antioxidants, preservatives, insect repellents, solubilizers, colorants, bacteria-inhibiting agents, hair conditioning agents, vitamins, and the like.

Examples of keratin adhesive and deposition aids include Allianz OPT, AquaStyle SH100, Conditioneze NT20-0, Aquaflex XL30, Silform INX, Celquat H100, Gransil SiW-038, Gransil SiW-MQIZ, and combinations of two or more of these aids. Keratin adhesive and deposition aids, when present, can each be present in any suitable amount, including from about 0.5% to about 5% by weight of the film forming compositions of the instant disclosure (e.g., about 1% to about 5%, about 1% to about 3%, about 0.5% to about 2%, 1% to about 2% or about 0.5% to about 1% by weight of the film forming compositions of the instant disclosure).

Reinforcement fillers include hydrophilic silica sold under the tradename SIPERNAT®, including SIPERNAT® 500LS. Reinforcement fillers, when present, can each be present in any suitable amount, including from about 0.01% to about 2% by weight of the film forming compositions of the instant disclosure (e.g., about 0.1% to about 0.5%, about 0.1% to about 0.3%, about 0.05% to about 0.5%, 0.01% to about 0.05% or about 0.01% to about 0.03% by weight of the film forming compositions of the instant disclosure).

The film forming compositions of the various embodiments described herein can be applied in any suitable fashion directly to the substrate using any suitable device (e.g., applied on sking using Swisspers® Wedges after face washed with tap water and dried).

In some embodiments film forming compositions can be applied following a pre-treatment of the substrate (e.g., keratinous tissue, including skin) to, among other things, improve adhesion to a substrate. In some embodiments, the substrate can be pre-treated with a suitable, non-toxic salt. While not wishing to be bound by any specific theory, it is believed that a salt solution can improve interfacial binding and/or increase binding sites between the resulting films and the substrate through salt bridging. For example a substantially aqueous solution comprising a salt, such as calcium chloride can be applied to the substrate by, e.g., finger with tapping. A film forming composition of the various embodiments described herein can then be applied. Suitable salts include, di- and tri-valent metal salts comprising suitable counterions. Suitable salts include but are not limited salts of multiple-valent metal ions including alkaline earth metal salts (e.g. Ca, Mg, Sr, Ba), transition metal salts (e.g. Ti, Mn, Fe, Cu, Zn, VV), and post transition metal salts (e.g. Al, Sn). Additional salts include salts such as aluminum chlorohydroxide (PACH) comprising a tri-valent aluminum metal PACH is an inorganic polymer with a broad range of variations. Due to its higher valence, PACH can effectively bridge product coatings to skin. Other aluminum salts, such as aluminum chloride and aluminum sulfate, are expected to provide similar benefits.

In still other embodiments, film forming compositions can be formed in situ by application on a substrate of the first copolymer and the second copolymer, separately, where the first copolymer can be applied to the substrate first, followed by application of the second copolymer or vice versa. In some embodiments, the first copolymer and the second copolymer can be applied simultaneously, but separately, using a dual-chamber applicator, where one chamber comprises a composition comprising the first copolymer and a second chamber comprises a composition comprising the second copolymer. In still other embodiments, the first copolymer and the second copolymer can be formulated as a foundation composition. The foundation composition can then be applied after applying a formulation comprising the first copolymer or the second copolymer matched such that a film forming composition according to the various embodiments described herein is obtained. In some embodiments, the second copolymer is applied to the substrate first, followed by the application of a foundation composition comprising the first copolymer.

In yet other embodiments, foundation can be applied directly on top of film forming compositions, as a top coat, before or after the film forming compositions are dry.

All percentages are by weight of the personal-care composition, unless otherwise specified. All ratios are weight ratios, unless specifically stated otherwise. All numeric ranges are inclusive of narrower ranges; delineated upper and lower range limits are interchangeable to create further ranges not explicitly delineated. The number of significant digits conveys neither limitation on the indicated amounts nor on the accuracy of the measurements. Unless otherwise stated or prescribed, all measurements are understood to be made from about 22-28° C. and at ambient conditions, where “ambient conditions” means conditions under about one atmosphere of pressure and at about 40-50% relative humidity.

“Keratinous tissue,” means keratin-containing tissue layers disposed as the outermost protective covering of mammals which includes, but is not limited to, skin, hair, and nails.

“Mascara” and “mascara composition” mean a liquid, semi-solid, or solid cosmetic composition that is applied to eyelashes to provide an aesthetic benefit or change in appearance such as, the appearance of a color change, a volume change, and/or a length change. Mascara may also be applied to periorbital areas, eyelids and/or eyebrows. The present mascara compositions are formulated for topical application to mammalian keratinous tissue for use in cosmetic products. The methods of using mascara compositions are also included within the meaning of mascara composition.

The term “foundation composition” generally means skin colored makeup applied to keratinous tissue (e.g., skin on the face) to create an even, uniform color to the complexion, to cover flaws and, sometimes, to change the natural skin tone. Some foundation compositions also function as a moisturizer, sunscreen, astringent or base layer for more complex cosmetics. Some foundation compositions can be applied to the body for decoration purposes.

Quaternary ammonium groups include pH dependent, such as primary, secondary, and tertiary amines, and pH independent, such as quaternized amines.

The ingredient % concentration and polymer ratios used herein are all based on active level or solid level.

“Pseudo cationic” generally means an electron-donor or proton acceptor molecule or functional group that is pH dependent, such as primary, secondary, and tertiary amines. At a pH above its isoelectric point, the molecule is non-ionic, while at pH below its isoelectric point, the molecule becomes cationic.

“Water-soluble, film-forming polymers” are defined herein to mean polymers which are soluble or dispersible in water, water-cosolvent mixtures (such as ethanol/water), pH adjusted water, and/or tempered solutions of the above to facilitate solubilization or dispersion of the polymers.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference.

In the methods described herein, the steps can be carried out in any order without departing from the principles of the disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

### EXAMPLES

The present disclosure can be better understood by reference to the following examples which are offered by way of illustration. The present disclosure is not limited to the examples given herein.

### Materials and Methods

Contraction Test

The principle of the contraction measurement is based on the shrinkage degree of a substrate after polymer compositions are applied to a substrate and dried.
Equipment

Leneta cards Form 2A (double coated opacity) with a dimension of 14 cm×25.4 cm, supplied by Leneta Company.

Single bar 3-inch Film Applicator, 6 mils thickness, supplied by BYK Gardner.

Drawdown Plate PA4200, supplied by BYK Gardner [0126] 4. Digital Humidity/Temperature Meter (Traceable® Model 35519-044 from VWR), or equivalent.

Digital balance (with minimum sensitivity of at least 0.001 g).

Measuring ruler (30 cm with mm scaling).
Procedure

Pre-weigh the Leneta card and record weight before a drawdown process. The drawdown method may refer ASTM D4062 or ASTM D2805 standard test methods. Position and secure the Leneta card on the Drawdown Plate. Place the bar film applicator centered at the top of the card, and load 5-10 grams of composition(s) comprising the first copolymer and the second copolymer distributed evenly across and immediately in front of the bar applicator. Ensure the amount of polymer composition load is sufficient to pass over the end of the card that gives a covered area of about 3 inches×8 inches by the casted film. Drawdown uniformly in the center of the card all the way down, and pass the polymer compositions to the end of the card and onto the drawdown plate. Ensure the casted film is evenly distributed and in an essentially rectangular shape. Allow the film to dry in a horizontal position for minimum 4 hours (typically overnight). Conduct experiments at a relative humidity of 40-50% and at a temperature in the range of 22° C. to 28° C. After the film is dried, the card is weighed again to determine the amount of total solids of the polymer compositions loaded by calculating the differences between the weights before and after the film cast.
Measurement and Calculation

Depending on the type of contraction or curling effect observed for a given composition on a card, only one of the calculation formulas given herein should be selected for best evaluations of the contraction under specified relative humidity and temperature conditions.

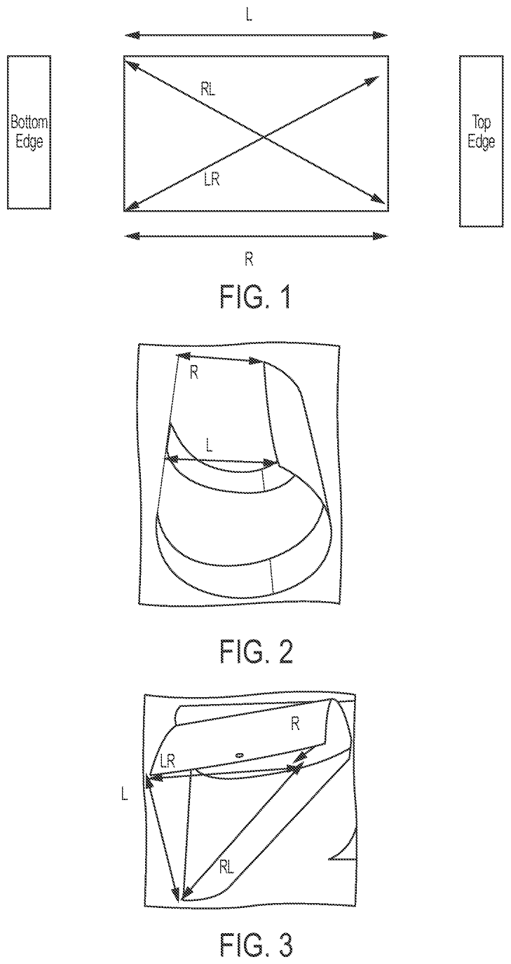

On a flat, untreated Leneta card (such as shown in FIG. 1), measure the distances (measured to nearest tenth of cm) from the top edge to bottom edge of the card in both right (R) and left (L) sides of the card. As shown in FIG. 1, L is the length on the left side of the card and R is the length on the right side of the card. Then, for an evenly contracted card (such as shown in FIG. 2), measure the L and R lengths after treatment with a polymer composition and allowing the card to dry. The degree of contraction (% Contraction) of the polymer composition is calculated as follows:

% Contraction=100×[1−(R+L)/(25.4×2)]

For example, if L=7.1 cm, and R=6.9 cm of an evenly contracted card (such as shown in FIG. 2), the % contraction is calculated below:

Contraction=100×[1−(6.9+7.1)/(25.4×2)]=72.4%

For non-even contraction or twisted cards, such as shown in FIG. 3, measure the distances (measured to nearest tenth of cm) from the top edge to bottom edge of the card in both right (R) and left (L) sides of the card, and also the distances of the diagonal of the card from the right top to left bottom (RL) and from the left top to right bottom (LR). The degree of contraction (% Contraction) of the polymer composition is calculated as follows:

% Contraction=100×[1−(R+L+RL+LR)/((25.4×2)+(29×2))]

For example, if L=5.8 cm, R=2.9 cm, LR=13.6, and RL=15.2 of a non-evenly contracted twisted card, the % Contraction is calculated below:

Contraction=100×[1−(5.8+2.9+13.6+15.2)/((25.4×2)+29×2))=65.5%

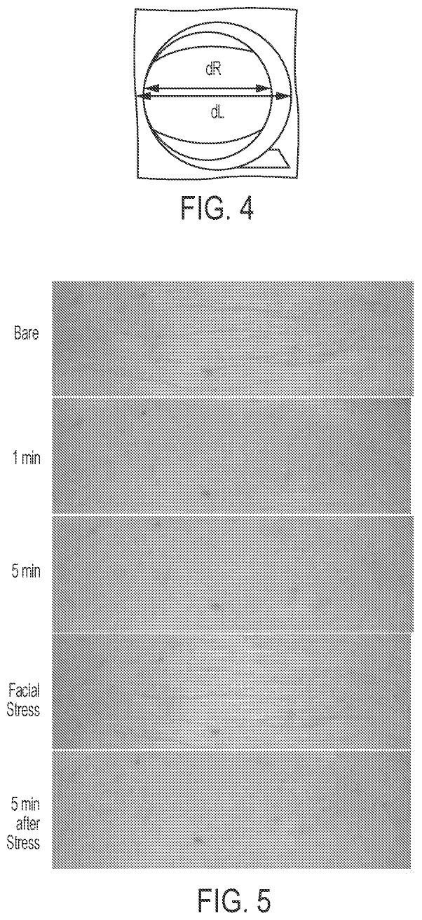

For coiled cards (such as shown in FIG. 4), measure diameters on both right (dR) and left (dL) edges (measure to nearest tenth of cm). The degree of contraction (% Contraction) of the card is calculated as follows:

Contraction=100×3.4218×(1/dR+1/dL)

For example, if dL=7.2 cm and dR=6.3 cm of a coiled card (where dL and dR are diameters measured from left side and right side respectively), the % Contraction is calculated below:

Contraction=100×3.4218×(1/7.2+1/6.3)=102%

The Leneta card Contraction Test is conducted primarily for polymer technical screening. The Leneta card method can be used in combinations with other contraction methods for contraction performances of polymer systems.

### Example 1

### In Vivo Image

The contraction synergy data was obtained in vivo on a subject's forehead. A formulation comprising 7.5% total polymers—a combination of Luviquat-Supreme and Luviset-One at a 2:1 ratio was applied on the forehead of a subject using Swisspers® Wedges after face washing with tap water and drying. Images were taken 1 and 5 minutes after application, then immediately after stretch and 5 minutes after stretching. The images are shown in FIG. 5. The images show significant tensioning benefits observed in less than ˜5 minutes. Significant recovery observed in ˜5 minutes after exaggerated facial stress.

### Example 2

### Contraction Synergy

Contraction synergy observed on Leneta card with two composition of the various embodiments described herein comprising 15% total polymers: B1 Luviquat-Supreme and Luviset-One at a 2:1 ratio; and B3: 15% total polymers Luviquat-Supreme and Luviset-Strong at a 2:1 ratio.

TABLE 1

| | B1 | B1 single | B3 | B3 single |
|---|---|---|---|---|
| | combination | Average | Combo | average |
| Contraction | 9.7% | 4.0% | 5.1% | 4.2% |

The data in Table 1 show a 245% synergy for B1 and 124% synergy for B3.

### Example 3

### Multiple-Valent Metal-Ion for Skin Adhesion Enhancement

One root cause of unmet consumer needs in long-wearing or performance durability with cosmetic or skincare products is insufficient skin adhesion due to weak interfacial binding or lack of binding sites between product films and skin. To gain active sites for more effective interactions, a ligand-metal complexation principle was applied to demonstrate that binding of the compositions described herein to the skin can be strengthened by what is believed to be a salt bridge at the molecular level.

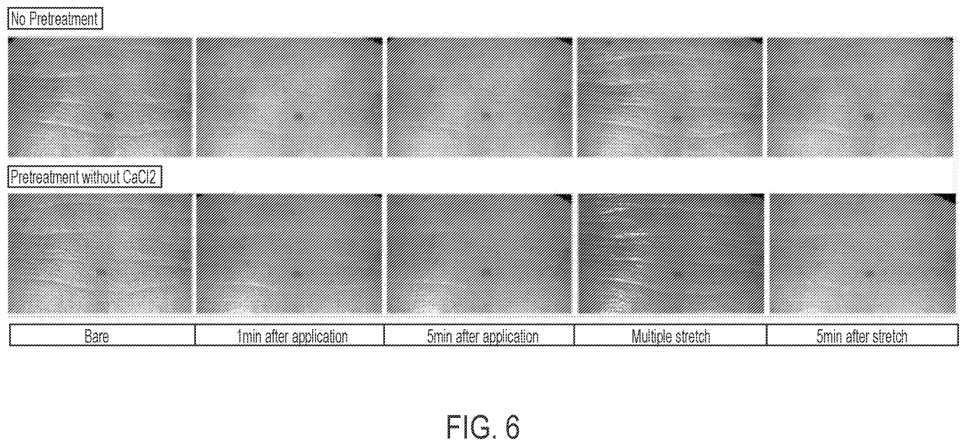

A composition according to the various embodiments described herein comprising Advantage-S (AdS) (5.00%), AquaStyle300 (A300) (5.00%), Sipernat 500LS (0.12%), AEROSIL® R816 (0.12%), BAYCUSAN® C1003 (1.50%), BAYCUSAN® C1005 (1.50%), ULTRATHIXTM P-100 (0.10%); and ethanol (1.50%) was applied on forehead with and without pre-treatment of CaCl₂ solution (about 1M in deionized water) by finger with tapping. The skin surface was evaluated. Images were taken 1 and 5 minutes after application, then immediately after stretch and 5 minutes after stretching. The images are shown in FIG. 6. The images show significant tensioning benefits observed in less than ˜5 minutes. Significant and improved recovery is observed in ˜5 minutes after exaggerated facial stress with CaCl₂ pretreatment. In addition, significant roughness reduction (pm) versus baseline (bare skin) was observed with CaCl₂ pretreatment, as shown in the data in Table 2

TABLE 2
Average surface roughness reduction

| | without CaCl₂ | with CaCl₂ |
|---|---|---|
| 1 min after application | −16.1 | −20.4 |
| 5 min after application | −16.5 | −22.4 |
| facial stretched | 17.8 | 10.1 |
| 5 min after stretched | −12.0 | −19.2 |

### Example 4

### Compositions for Skincare Cosmetics

Various compositions according to the various embodiments described herein were evaluated to determine, among other things, the effect of various additives on performance, flexibility, roughness/smoothness, and durability on those compositions. The compositions and results are shown in Tables 3-11.

TABLE 3

| | A300 | AdS | EtOH | 500LS | R816 | Flexibility | Roughness |
|---|---|---|---|---|---|---|---|
| D13 | 6.7% | 3.3% | 3.0% | 0.20% | 0.20% | + | − |
| D14 | 3.3% | 6.7% | 3.0% | 0.20% | 0.20% | ++ | −− |
| D15 | 2.50% | 2.50% | 3.0% | 0.10% | 0.10% | +++ | −− |
| D16 | 3.75% | 3.75% | 3.0% | 0.10% | 0.10% | + | + |

Flexibility Ranking:

| | | | |
|---|---|---|---|
| − | + | ++ | +++ |
| Crack- | Partial | Slight cracking | No cracking w/ |
| ing | cracking | w/bending | bending & high |
| w/ | w/bending | & high pres- | pressure squeeze |
| bending | & pressure | sure squeeze | |

Surface roughness Ranking:

| | | |
|---|---|---|
| − − | − | + |
| Very rough | Slightly rough | Smooth |

The data in Table 3 show, among other things under the test conditions and specific polymer combination, that lower polymer levels provided better film flexibility and durability; lower particle levels help reducing surface roughness; the polymer combination with cationic and anionic polymer ratio at 1:1 deliver better flexibility; and that the contraction of the polymer combination is less dependent of polymer ratios. While not wishing to be bound by any specific theory, it is believed that surface charge and molecular weight of the polymers may play some role in skin adhesion.

TABLE 4

| | R816 | PUR | C1000 | C1008 | C1004 | C2000 | OPT | NT20-0 | RE100 | SH100 | SA-N | EW | BioB | MadC | OugL | Flex | Smooth |
|---|---|---|---|---|---|---|---|---|---|---|---|---|---|---|---|---|---|
| A01 | 1.5% | | | | | | 1.0% | | | | | | | | | + | + |
| A02 | 1.5% | | | | | | | | 0.5% | | | | | | | −− | − |
| A03 | 1.5% | | | | | | | 1.0% | | | | | | | | −− | − |
| A04 | 1.5% | | | | | | | | | 1.0% | | | | | | − | − |
| A04 | 1.5% | | | | | | | | | | | 5% | | | | − | − |
| A04 | 1.5% | | | | | | | | | | 2.0% | | | | | − | − |
| B01 | 1.5% | | 5.0% | | | | | | | | | | | | | + | + |
| B02 | 1.5% | | | 5.0% | | | | | | | | | | | | + | + |
| B03 | 1.5% | | | | 5.0% | | | | | | | | | | | + | + |
| B04 | 1.5% | | | | | 5.0% | | | | | | | | | | −− | − |
| D01 | 0.2% | | | | | | | | | | | | | | | − | − |
| D02 | | 1.0% | | | | | | | | | | | | | | + | + |
| D03 | | | | | | | 1.0% | | | | | | | | | + | + |
| D04 | 0.2% | | | | | | 1.0% | | | | | | | | | + | + |
| D05 | | | | | 2.0% | | | | | | | | | | | ++ | + |
| D06 | 0.2% | | | | 5.0% | | | | | | | | | | | ++ | −− |
| D07 | | | | | | | | | | | | | 5.0% | | | + | − |
| D08 | 0.2% | | | | | | | | | | | | 5.0% | | | + | + |
| D09 | | | | | | | | | | | | | | 1.0% | | − | + |
| D10 | 0.2% | | | | | | | | | | | | | 1.0% | | + | + |
| D11 | | | | | | | | | | | | | | | 0.3% | + | + |
| D12 | 0.2% | | | | | | | | | | | | | | 0.3% | + | − |

PUR = Luviset PUR; C1000, C1008, C1004, and C2000 = BAYCUSAN ® C1000, C1008, C1004, and C2000, respectively; OPT = Allianz OPT; NT20-0 = Conditioneze NT20-0; RE100 = Si-Tec RE100; SH100 = AquaStyle SH100; SA-N = Biopolymer SA-N; EW = Neoppro EW; BioB = BioBenefity; MadC = MandarinClear; and OugL = Ougon Liquid B.

The data in Table 4 was generated using a base composition of 5% AdS, 5% A300, 3% ethanol, and 0.2% 500LS. Additional data were generated using a base composition of 5% AdS, 5% A300, and 2-3% ethanol, with fixed levels and equal ratios of 500LS/R816. Those data are shown in Table 5.

TABLE 5

| | EtOH | 500LS | R816 | BG | XL-30 | RE100 | SiW | Flexibility | Smoothness |
|---|---|---|---|---|---|---|---|---|---|
| D17 | 3.0% | 0.10% | 0.10% | | 2.00% | | | + | + |
| D20 | 3.0% | 0.10% | 0.10% | | | 2% | | −− | − |
| D21 | 3.0% | 0.10% | 0.10% | | | | 4.5% | + | − |
| D22 | 3.0% | 0.10% | 0.10% | 3.00% | | | | ++ | + |

BG = butylene glycol; and XL-30 = Aquaflex XL-30.

The data presented in Tables 4 and 5 show that polyurethane based elastomers, such as Luviset PUR and Baycusan C polymers, imbued the resulting films with significantly improved film flexibility and better durability. Among the elastomers, Baycusan C1004 delivered most flexibility benefits. In addition, butylene glycol also showed good elasticity that may contribute to flexibility of some bioactives such as plant extraction proteins.

The data shown in Tables 6-9 were generated with using a base composition of 5% ADS, 5% A300, and 2-3% ethanol, with fixed levels and equal ratios of 500LS/R816; a base composition of 5% AdS, 5% AdS, 0.025% 500LS, and 1.5% ethanol; a base composition of 2.5% ADS, 2.5% A300, 1.5-3% ethanol, with variable levels of 500LS/R816; and a base composition of 2.5% ADS, 2.5% A300, 0.025% 500LS, 0.025% R816, and 1.5% EtOH, respectively.

TABLE 6

| | EtOH | 500LS | R816 | BG | C1003 | C1004 | C1000 | P100 | Flexibility | Smoothness |
|---|---|---|---|---|---|---|---|---|---|---|
| D19 | 3.0% | 0.10% | 0.10% | | | | | 1.00% | − | + |
| D23 | 3.0% | 0.15% | 0.15% | | | 2.5% | | 0.50% | − | + |
| D24 | 3.0% | 0.15% | 0.15% | | | 5.0% | | 0.50% | ++ | + |
| D25 | 3.0% | 0.05% | 0.05% | 0.05% | | 2.5% | | 0.20% | ++ | − |
| D26 | 2.0% | 0.05% | 0.05% | | | 2.5% | | 0.10% | + | + |
| D27 | 2.0% | 0.05% | 0.05% | | 2.05% | | | 0.10% | +++ | + |
| D28 | 2.0% | 0.05% | 0.05% | | | | 2.50% | 0.10% | + | − |

P100 = ULTRATHIX ™ P-100

TABLE 7

| | PA | FA4103 | R816 | C1005 | P100 | AMP | Flexibility | Smoothness |
|---|---|---|---|---|---|---|---|---|
| C03 | 2.0% | | | 1.9% | 0.24% | 0.11% | ++ | − |
| C04 | | 2.0% | | 1.9% | 0.24% | 0.11% | + | − |
| C05 | | | 2.0% | 1.9% | 0.24% | 0.11% | − | − |

FA4103 = FA4103 silicone acrylate emulsion; and AMP = 2-Amino-2-Methyl-1-Propanol.

TABLE 8

| | EtOH | 500LS | R816 | BG | C1003 | C1004 | P100 | Flexibility | Roughness |
|---|---|---|---|---|---|---|---|---|---|
| D18 | 3.0% | 0.10% | 0.05% | | | 2.00% | | ++ | −− |
| D25d | 1.5% | 0.025% | 0.025% | 0.03% | | 1.25% | 0.10% | +++ | + |
| D29 | 1.50% | 0.025% | | | 2.0% | | 0.20% | +++ | − |

TABLE 9

| | C1003 | C1005 | 15D | SA | WS | PA | INX | H100 | MQIZ | SiW | P100 | Flexibility | Roughness |
|---|---|---|---|---|---|---|---|---|---|---|---|---|---|
| A01 | 2.0% | | 2.0% | | | | | | | | 0.2% | ++ | ++ |
| A02 | 2.0% | | | 2.0% | | | | | | | 0.2% | ++ | − |
| A03 | 2.0% | | | | 2.0% | | | | | | 0.2% | ++ | ++ |
| A04 | 2.0% | | | | | 2.0% | | | | | 0.2% | ++ | ++ |
| B05 | 2.0% | 2.0% | | | | | | | | | 0.2% | ++ | − |
| M06 | 2.0% | | | | | | 1.0% | | | | 0.2% | ++ | + |
| A07 | 2.0% | | | | | | | 1.0% | | | 0.2% | ++ | + |
| G08 | 2.0% | | | | | | | | 2.0% | | 0.2% | ++ | ++ |
| G09 | 2.0% | | | | | | | | | 2.0% | 0.2% | + | −− |

15D = Polynecon PPI-SA-15D; SA = Polyderm PPI-SA; WS = Polynecon PPI-SI-WS; PA = Polyderm PE-PA; INX = Silform INX; H100 = Celquat H100; MQIZ = Gransil SiW-MQIZ; and SiW = Gransil SiW-038.

The data in Tables 6-9 show, among other things, that UltraThix P100 was found surprisingly to improve flexibility of the polymer films; in-vivo results suggested a multiple polymer composition with UltraThix P100 delivered not only rheology modifications but also tensioning durability benefits; Baycusan C1003 demonstrated as the best elastomer under the test conditions to deliver significantly improved film durability; lower levels of total amounts of the first copolymer plus the second copolymer (e.g., 5% versus 10%) showed consistently better flexibility and durability through all the tests.

The data shown in Table 10 show, among other things, A300 and AdS as the first copolymer and the second copolymer, respectively, at 1:1 ratio (total 10%) among other things, delivered good tightening effect and durability. In addition, the data in Table 10 showed that a multiple polymer system, including a first copolymer and a second copolymer, polyurethane (PU) based elastomers and PU network polymers improved performance durability. Finally, a mixed particle system, including a hydrophilic precipitated silica, a hydrophobic fumed silica, and a synthetic PU particulate provides multiple functional synergy, such as film strength reinforcement, rheology modifications, and soft focus effects.

The data shown in Table 11 also show, among other things, that lower amounts of particles or no particles, including silica particles (both precipitated and fumed) and PU based optical particulate, help film transparency. As the result, there is less whiteness and better smoothening perception. In addition, the data in Table 11 show that PU based elastomer (like Baycusan C1003) at relatively high levels (1-2%) improves durability, although there may be a gain in opaqueness of the film; PVP/acrylate cross-polymer provides sufficient tightening benefits (both immediate and durable) at lower amounts (0.1-0.2%); and lower pH provides good skin feel, even though it may impact negatively effectiveness of PVP/acrylate cross polymer for durability benefits.

### Example 5

### Application Methods for Skincare Cosmetics

Various compositions according to the various embodiments described herein were evaluated in terms of their suitability as face-masks, two-component systems, and makeup regimen applications.

For example, a composition according to the various embodiments described herein comprising AdS (5.00%), A300 (5.00%), Sipernat 500LS (1%), AEROSIL® R816 (1%), and ethanol (3%) was applied via a facemask. Preliminary results show improved durability with facemask likely due to enhanced skin adhesion. Increased skin/film interactions with facemask was believed to be contributed by longer skin contact time and delayed polymer contractions due to longer solution phase or slower wet film phase transition (slower evaporation). In addition, facemask may help in better control of film thickness and dosing uniformity across face locations.

The compositions of the various embodiments described herein can also be formulated into two-component composition systems, where one component comprises the first copolymer (e.g., 5.00% A300 plus 2.00% 500LS) and the other component comprises the second copolymer (e.g., 5.00% AdS plus 1.00% R816). One of the components is applied first, followed by application of another component. Preliminary data showed improved durability with two-step applications. Interference of cohesive (network contraction) on skin adhesion is thus minimized.

In makeup regimen applications, two application methods were developed: the dual-chamber one-step application and the lock-and-key two-step application.

In the context of dual-chamber one-step application, premixing a composition according to the various embodiments described herein with a foundation prior to skin applications delivered smoother appearance as demonstrated by image data (not shown). In addition, controlled dosing by a dual chamber is expected to minimize color variations. This may help in final shade definition for consumers.

In the context of lock-and-key two-step application, a first copolymer and a second copolymer were formulated separately—one in a film forming primer composition and the other in a foundation composition. The method provides freedom to select a right polymer in a different composition and corresponding application steps. Significant tensioning/smoothing benefits were observed by two step applications. The data suggest that the two polymers can be incorporated in any component without loss in observed benefits. In addition to the smoothening and tension benefits of foundations, such two-component approaches are cost effective (e.g., no need a more expensive devise for premixing), easy to use for consumers (e.g., no need for time control, and no need to worry about compression pressure).

TABLE 10

| | | | | | | | | | | Vis | | |
|---|---|---|---|---|---|---|---|---|---|---|---|---|
| | A300 | AdS | 500LS | R816 | C1003 | C1005 | P100 | AMP | pH | (mPa) | Tightening | Durability |
| C01 | 2.5% | 2.5% | 0.025% | 0.025% | 2.0% | | 0.2% | 0.097% | 8.21 | 3000 | + | + |
| C02 | 5.0% | 5.0% | | | 2.0% | 2.0% | 0.2% | 0.097% | 8.60 | 4000 | ++ | − |
| C06 | 5.0% | 5.0% | 0.15% | 0.15% | 1.5% | 1.0% | 0.1% | 0.038% | 8.48 | 1600 | ++ | − |
| C07 | 4.0% | 4.0% | 0.15% | 0.15% | 1.5% | 1.0% | 0.15% | 0.058 | 8.21 | 2100 | ++ | − |
| C08 | 4.0% | 4.0% | 0.12% | 0.12% | 1.0% | 1.5% | 0.15% | | 7.06 | 1600 | ++ | + |
| C09 | 5.0% | 5.0% | 0.12% | 0.12% | 1.5% | 1.5% | 0.10% | | | | ++ | + |

TABLE 11

| | A300 | AdS | 500LS | R816 | C1003 | C1005 | P100 | EtOH | pH | mPa | Flexibility | Roughness | Transparency |
|---|---|---|---|---|---|---|---|---|---|---|---|---|---|
| C02 | 5.00% | 5.00% | | | 2.00% | 2.00% | 0.20% | 1.50% | 8.6 | 4000 | ++ | − | − |
| C05 | 5.00% | 5.00% | 0.12% | 0.12% | | 2.00% | 1.50% | 1.50% | 8.51 | 2400 | + | − | + |
| C08 | 4.00% | 4.00% | 0.12% | 0.12% | 1.00% | 1.50% | 0.15% | 1.50% | 7.06 | 1600 | ++ | − | − |

All documents cited herein are, in relevant part, incorporated herein by reference; the citation of any document is not to be construed as an admission that it is prior art with respect to the present disclosure. To the extent that any meaning or definition of a term in this document conflicts with any meaning or definition of the same term in a document incorporated by reference, the meaning or definition assigned to that term in this document shall govern.

The present disclosure provides for the following embodiments, the numbering of which is not to be construed as designating levels of importance:

Embodiment 1 relates to a film forming composition comprising: a first anionic, pseudo-cationic or non-ionic non-crosslinking polyacrylate random copolymer comprising the following monomer units:
at least one acrylate monomer, including acrylate, (meth)acrylate monomers or short-chain alkyl acrylate;
monomers having at least one carboxylic functional group selected from the group consisting of carboxylic esters, carboxylic acids, their salts, or precursors of carboxylate functions, and mixtures thereof and
a second cationic or pseudo-cationic non-crosslinking polyamide random copolymer comprising the following monomer units:
at least one amide monomer, including vinyl caprolactam monomers, vinylpyrrolidone monomers, and (meth)acrylamide or short-chain alkyl acrylamide monomers;
at least one cationic functional group, including quaternary amine or ammonium containing monomer and monomers having an amine functional group including primary, secondary, and tertiary amines;
wherein:
the film forming composition is a combination of first and second polymers; and if the second copolymer has a charge density of about 0.1 mEq/g to about 1 mEq/g, the first copolymer has a charge density of about −1 mEq/g to about −6 mEq/g or if the second copolymer has a charge density of about 1 mEq/g to about 6 mEq/g, the first copolymer has a charge density of about −0.1 mEq/g to about −1 mEq/g.

Embodiment 2 relates to the composition of Embodiment 1, wherein said first and second copolymers comprise less than 40% of the same monomers.

Embodiment 3 relates to the composition of Embodiments 1-2, wherein said first and second copolymers comprise from about 0% to about 39% of the same monomers.

Embodiment 4 relates to the composition of Embodiments 1-3, wherein said first and second copolymers have from about 0% to about 20% of the same monomers.

Embodiment 5 relates to the composition of Embodiments 1-4, wherein said first and second copolymers have from about 0% to about 10% of the same monomers.

Embodiment 6 relates to the composition of Embodiments 1-5, wherein said second copolymer has one or more quaternary ammonium or other cationic side chains and further, wherein said second polymer comprises from about 0.1 to about 45 percent of said quaternary ammonium or other cationic side chains.

Embodiment 7 relates to the composition of Embodiments 1-6, wherein said second copolymer has one or more quaternary ammonium or other cationic side chains and further, wherein said second polymer comprises from about 1 to about 20 percent of said quaternary ammonium or other cationic side chains.

Embodiment 8 relates to the composition of Embodiments 1-7, wherein said first copolymer has one or more carboxylate or other anionic side chains and further, wherein said first polymer comprises from about 0.1 to about 45 percent of said carboxylate or other anionic side chains.

Embodiment 9 relates to the composition of Embodiments 1-8, wherein said first copolymer has one or more carboxylate or other anionic side chains and further, wherein said first polymer comprises from about 1 to about 30 percent of said carboxylate or other anionic side chains.

Embodiment 10 relates to the composition of Embodiments 1-9, wherein the ratio of said first copolymer to said second copolymer is from about 1:50 to about 50:1.

Embodiment 11 relates to the composition of Embodiments 1-10, wherein the ratio of said first copolymer to said second copolymer is from about 1:10 to about 10:1.

Embodiment 12 relates to the composition of Embodiments 1-11, wherein the ratio of said first copolymer to said second copolymer is about 1:3 to about 3:1.

Embodiment 13 relates to the composition of Embodiments 1-12, wherein the ratio of said first copolymer to said second copolymer is about 1:2 to about 2:1.

Embodiment 14 relates to the composition of Embodiments 1-13, wherein the composition produces a minimum contraction of about 5% to about 10% with minimum synergy of about 120% to about 160% when applied to a Leneta card.

Embodiment 15 relates to the composition of Embodiment 1-14, further comprising one or more polyurethane based elastomers.

Embodiment 16 relates to the composition of Embodiments 1-15, further comprising one or more acrylic acid/vinylpyrrolidone crosspolymers.

Embodiment 17 relates to the composition of Embodiments 1-16, wherein the amount of total polymers in the composition is from about 1% to about 30%.

Embodiment 18 relates to the composition of Embodiments 1-17, further comprising a mixed particle system comprising at least one of a hydrophilic precipitated silica and a hydrophobic fumed silica.

Embodiment 19 relates to the composition of Embodiments 1-18, wherein the composition is in the form of a liquid film, a dry film, a dry powder, or combinations thereof.

Embodiment 20 relates to a water based system comprising the film forming composition of Embodiments 1-19.

Embodiment 21 relates to a mascara formulation comprising the composition of Embodiments 1-20.

Embodiment 22 relates to a skin care product comprising the composition of Embodiments 1-20.

Embodiment 23 relates to a skin foundation product comprising the composition of Embodiments 1-20.

Embodiment 24 relates to a primer product comprising the composition of Embodiments 1-20.

Embodiment 25 relates to a hair care product comprising the composition of Embodiments 1-20.

Embodiment 26 relates to a body care product comprising the composition of Embodiments 1-20.

Embodiment 27 relates to a facemask having a first major surface and a second major surface opposite the first major surface, wherein at least a portion of the second major surface comprising the composition of Embodiments 1-20.

Embodiment 28 relates to a method of applying the composition of Embodiments 1-20 to a substrate, the method comprising pre-treating the substrate with an aqueous solution, aqueous suspension or water-based emulsion comprising a metal-ligand complex; and subsequently applying the composition of Embodiments 1-20.

Embodiment 29 relates to a two-component system comprising:
as a first component, a first anionic or non-ionic non-crosslinking polyacrylate random copolymer comprising the following monomer units:
at least one acrylate monomer, including acrylate, (meth)acrylate monomers or short-chain alkyl acrylate;
monomers having at least one carboxylic functional group selected from the group consisting of carboxylic esters, carboxylic acids, their salts, or precursors of carboxylate functions, and mixtures thereof.
as a second component separate from the first component, a second cationic non-crosslinking polyamide random copolymer comprising the following monomer units:
at least one amide monomer, including vinyl caprolactam monomers, vinylpyrrolidone monomers, and (meth)acrylamide or short-chain alkyl acrylamide monomers;
at least one cationic functional group, including quaternary amine or ammonium containing monomer and monomers having an amine functional group including primary, secondary, and tertiary amines.

Embodiment 30 relates to the two-component system of Embodiment 29, wherein the first component or the second component is a foundation composition.

Embodiment 31 relates to the two-component system of Embodiment 29, wherein the first component or the second component is a primer composition.

Embodiment 32 relates to a method of applying the two-component system of Embodiment 29 comprising applying the first component first and subsequently applying the second component or applying the second component first and subsequently applying the first component.

Embodiment 33 relates to a dual-chamber applicator comprising a first chamber and a second chamber, wherein the first chamber comprises the first component of Embodiment 29 and the second chamber comprises the second component of Embodiment 29.

1. A film forming composition comprising:
a first anionic, pseudo-cationic or non-ionic non-crosslinking polyacrylate random copolymer comprising the following monomer units:
at least one acrylate monomer, including acrylate, (meth)acrylate monomers or short-chain alkyl acrylate;
monomers having at least one carboxylic functional group selected from the group consisting of carboxylic esters, carboxylic acids, their salts, or precursors of carboxylate functions, and mixtures thereof and
a second cationic or pseudo-cationic non-crosslinking polyamide random copolymer comprising the following monomer units:
at least one amide monomer, including vinyl caprolactam monomers, vinylpyrrolidone monomers, and (meth)acrylamide or short-chain alkyl acrylamide monomers;
at least one cationic functional group, including quaternary amine or ammonium containing monomer and monomers having an amine functional group including primary, secondary, and tertiary amines.

2. The composition of claim 1, wherein the composition is a two component system comprising:
as a first component, a first anionic or non-ionic non-crosslinking polyacrylate random copolymer comprising the following monomer units:
at least one acrylate monomer, including acrylate, (meth)acrylate monomers or short-chain alkyl acrylate;
monomers having at least one carboxylic functional group selected from the group consisting of carboxylic esters, carboxylic acids, their salts, or precursors of carboxylate functions, and mixtures thereof and
as a second component separate from the first component, a second cationic non-crosslinking polyamide random copolymer comprising the following monomer units:
at least one amide monomer, including vinyl caprolactam monomers, vinylpyrrolidone monomers, and (meth)acrylamide or short-chain alkyl acrylamide monomers;
at least one cationic functional group, including quaternary amine or ammonium containing monomer and monomers having an amine functional group including primary, secondary, and tertiary amines.

3. The composition of claim 1, wherein the film forming composition is a combination of first and second polymers; and
if the second copolymer has a charge density of about 0.1 mEq/g to about 1. mEq/g, the first copolymer has a charge density of about −1 mEq/g to about −6 mEq/g or if the second copolymer has a charge density of about 1 mEq/g to about 6 mEq/g, the first copolymer has a charge density of about −0.1 mEq/g to about −1 mEq/g.

4. The composition of claim 3, wherein said first and second copolymers comprise less than 40% of the same monomers.

5. The composition of claim 3, wherein the second copolymer has one or more quaternary ammonium or other cationic side chains and fitrther, wherein said second polymer comprises from about 0.1 to about 45 percent of said quaternary ammonium or other cationic side chains; or
wherein the first copolymer has one or more carboxylate or other anionic side chains and further, wherein said first polymer comprises from about 0.1 to about 45 percent of said carboxyl ate or other anionic side chains.

6. The composition of claim 3, wherein the ratio of said first copolymer to said second copolymer is from about 1:10 to about 10:1; or
the ratio of said first copolymer to said second copolymer is about 1:2 to about 2:1.

7. The composition of claim 3, wherein the composition produces a minimum contraction of about 5% to about 10% with minimum synergy of about 120% to about 160% when applied to a Leneta card.

8. The composition of claim 3, further comprising one or more polyurethane based elastomers; or
one or more acrylic acid/vinylpyrrolidone crosspolymers.

9. The composition of claim 3, wherein the amount of total polymers in the composition is from about 1% to about 30%.

10. The composition of claim 3, further comprising a mixed particle system comprising at least one of a hydrophilic precipitated silica and a hydrophobic fumed silica.

11. The composition of claim 3, wherein the composition is in the form of a liquid film, a dry film, a dry powder, or combinations thereof.

12. A water based system rising the film forming composition of claim 3.

13. A mascara formulation, skin care product, skin foundation product, primer product, hair care product or body care product comprising the composition of claim 3.

14. A facemask having a first major surface and a second major surface opposite the first major surface, wherein at least a portion of the second major surface comprising the composition of claim 3.

15. A dual-chamber applicator comprising a first chamber and a second chamber, wherein the first chamber comprises the first component of claim 2 and the second chamber comprises the second component of claim 2.
